(19) United States
(12) Patent Application Publication
(10) Pub. No.: US 2011/0150801 A1
(43) Pub. Date: Jun. 23, 2011

(54) COLOURED COMPOSITION FOR CONCEALING SKIN IMPERFECTIONS

(75) Inventor: Caroline LEBRE-LEMONNIER, Paris (FR)

(73) Assignee: L'OREAL, Paris (FR)

(21) Appl. No.: 12/969,661

(22) Filed: Dec. 16, 2010

Related U.S. Application Data
(60) Provisional application No. 61/290,962, filed on Dec. 30, 2009.

(30) Foreign Application Priority Data
Dec. 18, 2009 (FR) 09 59221

(51) Int. Cl. A61K 8/19; A61Q 1/02

(52) U.S. Cl. 424/63

(57) ABSTRACT

A coloured composition useful for making up keratinous substances, in particular the skin, in the form of a water-in-oil emulsion containing at least 18% by weight of pulverulent colouring materials, with respect to the total weight of the composition, and at least one or more volatile linear alkanes, in particular C₇-C₁₄ alkanes. Methods of use.

### REFERENCE TO PRIOR APPLICATIONS

This application claims priority to U.S. provisional application Ser. No. 61/290,962, filed Dec. 30, 2009; and to French patent application 09 59221, filed Dec. 18, 2009, both incorporated herein by reference.

### FIELD OF THE INVENTION

A subject-matter of the present invention is a coloured composition preferably useful for making up keratinous substances, in particular the skin, in the form of a water-in-oil emulsion capable of concealing colour imperfections of the said keratinous substances. The compositions concerned are in particular foundations or complexion correctors.

The term “coloured composition” is understood to mean a nonwhite composition.

### BACKGROUND OF THE INVENTION

To obtain a makeup result which makes it possible to conceal skin imperfections, such as blemishes or hypo- or hyperpigmented areas, generally involves the incorporation of a high level of pigments, which is very often reflected by undesirable applicational properties: the texture of the composition becomes pasty and thus difficult to spread. The makeup obtained is not homogeneous and areas with different colours appear on the skin, rendering the makeup unsightly. Furthermore, the pasty texture is uncomfortable for the user during and after the application of the composition to the skin.

Although silicone oils made it possible about 20 years ago to advance the sensoriality of products of foundation type, the preparation of such products, which are covering but pleasant to apply, often proves to be difficult.

The preparation of pigmented emulsions with a high covering power represents in particular a difficult technical challenge to be taken up by the formulator. This is because high coverage and pleasant cosmetic properties are very often two antagonistic parameters difficult to reconcile, all the more so as the high level of pigments increases the problems of stability of the composition during the storage thereof over time, this being all the truer when the composition is in the form of an emulsion. This is because the pigments have a tendency to sediment and a significant supernatant of oils may appear. The composition then exhibits phase separation and is no longer homogeneous: it does not make it possible to obtain a homogeneous makeup and its appearance becomes particularly unsightly.

It is often easier to obtain an opacifying makeup film via anhydrous formulation. However, the latter exhibits the disadvantage of not making possible the ready incorporation of the active principles used in cosmetics (antiwrinkle active principles, moisturising active principles, intended for greasy skin, and the like), which are generally dissolved in the aqueous phase of the preparation. Furthermore, anhydrous complexion products are not appreciated by all types of consumers, given their sometimes greasy or oily nature.

There thus exists a need to have available makeup compositions which make it possible to effectively conceal colour defects of the skin (in particular blemishes and hypo- or hyperpigmented areas) and which exhibit a texture which makes it possible for easy application to the skin.

### DETAILED DESCRIPTION OF THE PREFERRED EMBODIMENTS

The inventor has discovered that the use of one or more volatile linear C₇-C₁₄ alkanes in the oily phase of a water-in-oil emulsion makes it possible to incorporate a high content of pigments in the emulsion (indeed even contents of greater than 25% by weight) and gives the product obtained improved cosmetic properties on application (which the use of silicone oils does not always make possible).

More specifically, the present invention is targeted at a composition for making up keratinous substances, in particular the skin, in the form of a water-in-oil emulsion, which makes it possible to effectively and homogeneously cover skin imperfections, in particular colour defects of the skin, while retaining a fluid texture, which is pleasant to use and which results in a makeup exhibiting good coverage.

According to a first aspect, a subject-matter of the invention is a coloured composition for making up keratinous substances, intended in particular to conceal skin imperfections, in particular colour defects of the skin, in the form of a water-in-oil emulsion comprising, in a physiologically acceptable medium:

(i) at least 18% by weight of one or more pulverulent colouring materials, with respect to the total weight of the composition,

(ii) at least one or more volatile linear alkane(s), in particular C₇-C₁₄ alkane(s).

The pulverulent colouring materials used according to the invention preferably have a size of greater than or equal to 100 nm.

In particular, the pulverulent colouring materials are preferably chosen from particles having a size ranging from 100 nm to 10 μm, more preferably from 200 nm to 5 μm and even more preferably from 250 nm to 1 μm.

Preferably, the said pulverulent colouring materials comprise at least coloured (nonwhite) particles, in particular iron oxides.

According to a preferred form, the pulverulent materials comprise iron oxides and titanium oxides.

In particular, the volatile linear alkane or alkanes, in particular C₇-C₁₄ alkane or alkanes, preferably represent at least 50% by weight, in particular at least 65% by weight, especially at least 80% by weight, indeed even 100% by weight, with respect to the total weight of the oils of the composition.

According to a specific form, the composition of the invention comprises at least 20% by weight, in particular at least 25% by weight, indeed even at least 30% by weight, of pulverulent colouring materials, with respect to the total weight of the said composition.

The composition according to the invention is in particular a foundation or a complexion corrector. It can be applied uniformly to the skin of the face or in small dabs in order to conceal skin imperfections. It will then be a complexion corrector.

The composition of the invention generally exhibits a coverage of greater than 50, preferably greater than 60 and better still of greater than 70. In particular, it exhibits a coverage ranging from 70 to 100, in particular from 80 to 100 and better still from 90 to 100.

Measurement of Coverage

The substrates used for the measurement of coverage according to the invention are Bioskin sheets white and black in colour (supplier Maprecos). The compositions according to the invention are applied with a fingerstall at a rate of 32 mg to a surface with a diameter of 5 cm and are then dried for 90 minutes. In particular, the thickness applied is 15 μm.

The reflectance spectra are acquired using a Minolta 3700-d spectrocolorimeter (diffuse measurement geometry and D65/10° observation, specular component mode excluded) on black and white backgrounds (three measurements on each application).

The spectra are expressed as colorimetric coordinates in the CIE Yxy 1931 space within the meaning of the International Commission on Illumination.

The contrast ratio CR, or coverage, is calculated by taking the arithmetic mean of Y on the black background, dividing by the mean of Y on the white background and multiplying by 100.

The present invention is also targeted at a cosmetic method for making up keratinous substances, in particular the skin, comprising the application of a composition according to the invention.

In particular, the said method is intended to conceal skin imperfections, in particular colour defects of the skin.

The composition according to the invention is in the form of a water-in-oil emulsion comprising an aqueous phase dispersed in a liquid fatty phase and comprises at least one physiologically acceptable medium.

The term “physiologically acceptable medium” is intended to denote a medium suitable in particular for the application of a composition of the invention to keratinous substances, in particular the skin and lips.

The physiologically acceptable medium is generally suited to the nature of the substrate on which the composition has to be applied and to the appearance under which the composition has to be packaged.

The composition according to the invention comprises at least 18% by weight of pulverulent colouring materials, with respect to the total weight of the said composition.

Pulverulent Colouring Materials

The pulverulent colouring materials can be present in a proportion of 18 to 70% by weight, in particular of 20 to 50% by weight and especially of 25 to 35% by weight, with respect to the total weight of the cosmetic composition.

According to a preferred form, the pulverulent colouring materials comprise at least coloured (nonwhite) particles.

The pulverulent colouring materials are chosen in particular from pigments, pearlescent agents, materials with a specific optical effect, and their mixtures. In particular, they will be pigments.

Pigments

The term “pigments” should be understood as meaning white or coloured and inorganic or organic particles which are insoluble in an aqueous solution and which are intended to colour and/or opacify the resulting film.

In particular, the composition according to the invention comprises at least coloured particles, in particular iron oxides.

Mention may be made, as inorganic pigments which can be used in the invention, of titanium, zirconium or cerium oxides and also zinc, iron or chromium oxides, ferric blue, manganese violet, ultramarine blue and chromium hydrate.

Use is preferably made of pigments formed of iron oxides, titanium dioxide and their mixtures.

The pigment can also have a structure which can, for example, be of sericite/brown iron oxide/titanium dioxide/silica type. Such a pigment is sold, for example, under the reference Coverleaf NS or JS by Chemicals and Catalysts and exhibits a contrast ratio in the region of 30.

The colouring material can also comprise a pigment having a structure which can, for example, be of the type of silica microspheres comprising iron oxide. An example of a pigment exhibiting this structure is that sold by Miyoshi under the reference PC Ball PC-LL-100 P, this pigment being composed of silica micro-spheres comprising yellow iron oxide.

Mention may be made, among the organic pigments which can be used in the invention, of carbon black, pigments of D & C type, lakes based on cochineal carmine of barium, strontium, calcium or aluminium, or the diketopyrrolopyrroles (DPP) described in the documents EP-A-542 669, EP-A-787 730, EP-A-787 731 and WO-A-96/08537.

Advantageously, the pigments can be present in a hydrophobic coated form in the emulsion according to the invention. They are more particularly pigments treated at the surface with a hydrophobic agent in order to render them compatible with the fatty phase of the emulsion, in particular in order for them to have a good wettability with the oils of the fatty phase. Thus, these treated pigments are well dispersed in the fatty phase.

The hydrophobic treatment agent can be chosen from silicones, such as methicones, dimethicones or perfluoroalkylsilanes; fatty acids, such as stearic acid; metal soaps, such as aluminium dimyristate or the aluminium salt of hydrogenated tallow glutamate; perfluoroalkyl phosphates, perfluoroalkylsilanes, perfluoroalkylsilazanes, poly(hexafluoropropylene oxide)s, polyorganosiloxanes comprising perfluoroalkyl perfluoropolyether groups, amino acids; N-acylated amino acids or their salts; lecithin, isopropyl titanium triisostearate, and their mixtures.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be aluminium, magnesium, calcium, zirconium, zinc, sodium or potassium salts. The amino acid can, for example, be lysine, glutamic acid or alanine.

The term “alkyl” mentioned in the abovementioned compounds denotes in particular an alkyl group having from 1 to 30 carbon atoms and preferably having from 5 to 16 carbon atoms.

Hydrophobic treated pigments are described in particular in Application EP-A-1 086 683.

Pearlescent Agents

The term “pearlescent agents” should be understood as meaning coloured particles of any shape, which may or may not be iridescent, produced in particular by certain shellfish in their shells or synthesized, which exhibit a colouring effect by optical interference.

The pearlescent agents can be chosen from pearlescent pigments, such as titanium oxide-coated mica covered with an iron oxide, titanium oxide-coated mica covered with bismuth oxychloride, titanium oxide-coated mica covered with chromium oxide or titanium oxide-coated mica covered with an organic dye, and pearlescent pigments based on bismuth oxychloride. They can also be mica particles, at the surface of which at least two successive layers of metal oxides and/or of organic colouring materials are superimposed.

Mention may also be made, as examples of pearlescent agents, of natural mica covered with titanium oxide, iron oxide, natural pigment or bismuth oxychloride.

Materials with an Optical Effect

The cosmetic composition according to the invention can also comprise at least one material with a specific optical effect.

This effect is different from a simple conventional colouring effect, that is to say a unified and stabilized effect, such as that produced by conventional colouring materials, such as, for example, monochromatic pigments. Within the meaning of the invention, the term “stabilized” means devoid of an effect of variability in the colour with the angle of observation or else in response to a change in temperature.

For example, this material can be chosen from particles with a metallic glint, goniochromatic colouring agents, diffracting pigments, thermochromic agents, optical brighteners and fibres, in particular interference fibres. Of course, these various materials can be combined so as to provide the simultaneous display of two effects, indeed even of a novel effect in accordance with the invention.

The particles with a metallic glint which can be used in the invention are chosen in particular from:
particles of at least one metal and/or of at least one metal derivative,
particles comprising an organic or inorganic substrate, made of one or more materials, at least partially covered with at least one layer with a metallic glint comprising at least one metal and/or at least one metal derivative, and
mixtures of the said particles.

Mention may be made, among the metals which can be present in the said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example, bronzes and brasses) are preferred metals.

The term “metal derivatives” denotes compounds derived from metals, in particular oxides, fluorides, chlorides and sulphides.

The goniochromatic colouring agent can be chosen, for example, from interference multilayer structures and liquid crystal colouring agents.

The composition according to the invention comprises at least one or more volatile linear alkanes, in particular C₇-C₁₄ alkanes.

Volatile Linear Alkanes

The composition of the invention can comprise from 5 to 50% by weight of volatile linear alkane(s), in particular from 8 to 40% by weight of volatile linear alkane(s) and more particularly from 10 to 30% by weight of volatile linear alkane(s), in particular C₇-C₁₄ alkane(s), with respect to the total weight of the composition.

According to a specific embodiment, the composition of the invention can comprise at least 50% by weight of volatile linear alkane(s), with respect to the total content of volatile oil(s) of the composition. In particular, a composition of the invention can comprise at least 60%, more particularly at least 70%, and more particularly at least 80%, at least 90% or 100% of volatile linear alkane(s), with respect to the total content of volatile oil(s) of the composition.

A composition of the invention comprising 100% of volatile linear alkane(s), with respect to the total content of volatile oil(s) comprises a volatile oily phase composed exclusively of volatile linear alkane(s).

The term “oil” is understood to mean a water-immiscible nonaqueous compound which is liquid at ambient temperature (25° C.) and atmospheric pressure (760 mmHg).

The term “hydrocarbon oil” is understood to mean an oil essentially formed, indeed even composed, of carbon and hydrogen atoms and optionally of oxygen and nitrogen atoms which does not comprise a silicon or fluorine atom; it can comprise ester, ether, amine and amide groups.

The term “one or more volatile linear alkane(s)” is understood to mean, without distinction, “one or more volatile linear alkane oil(s)”.

A volatile linear alkane suitable for the invention is liquid at ambient temperature (approximately 25° C.) and at atmospheric pressure (760 mmHg).

The term “volatile linear alkane” suitable for the invention is understood to mean a cosmetic linear alkane capable of evaporating on contact with the skin in less than one hour at ambient temperature (25° C.) and atmospheric pressure (760 mmHg, that is to say 101 325 Pa), which is liquid at ambient temperature and which has in particular a rate of evaporation ranging from 0.01 to 15 mg/cm²/min at ambient temperature (25° C.) and atmospheric pressure (760 mmHg).

Preferably, the “volatile linear alkanes” suitable for the invention exhibit a rate of evaporation ranging from 0.01 to 3.5 mg/cm²/min at ambient temperature (25° C.) and atmospheric pressure (760 mmHg).

Preferably, the “volatile linear alkanes” suitable for the invention exhibit a rate of evaporation ranging from 0.01 to 1.5 mg/cm²/min at ambient temperature (25° C.) and atmospheric pressure (760 mmHg).

More preferably, the “volatile linear alkanes” suitable for the invention exhibit a rate of evaporation ranging from 0.01 to 0.8 mg/cm²/min, at ambient temperature (25° C.) and atmospheric pressure (760 mmHg).

More preferably, the “volatile linear alkanes” suitable for the invention exhibit a rate of evaporation ranging from 0.01 to 0.3 mg/cm²/min, at ambient temperature (25° C.) and atmospheric pressure (760 mmHg).

More preferably, the “volatile linear alkanes” suitable for the invention exhibit a rate of evaporation ranging from 0.01 to 0.12 mg/cm²/min at ambient temperature (25° C.) and atmospheric pressure (760 mmHg).

The rate of evaporation of a volatile alkane in accordance with the invention (and more generally of a volatile solvent) can be evaluated in particular by means of the protocol described in WO 06/013413 and more particularly by means of the protocol described below.

15 g of volatile hydrocarbon solvent are introduced into a crystallizing dish (diameter: 7 cm) placed on a balance located in a chamber with a capacity of approximately 0.3 m³ regulated with regard to temperature (25° C.) and hygrometry (relative humidity 50%).

The liquid is allowed to freely evaporate, without being stirred, ventilation being provided by a fan (Papst-Motoren, reference 8550 N, rotating at 2700 revolutions/minute) arranged in the vertical position above the crystallizing dish containing the volatile hydrocarbon solvent, the blades being directed towards the crystallizing dish, at a distance of 20 cm with respect to the bottom of the crystallizing dish.

The weight of volatile hydrocarbon solvent remaining in the crystallizing dish is measured at regular time intervals.

The evaporation profile of the solvent is then obtained by plotting the curve of the amount of product evaporated (in mg/cm²) as a function of the time (in min).

The rate of evaporation, which corresponds to the tangent at the origin of the curve obtained, is then calculated. The rates of evaporation are expressed in mg of volatile solvent evaporated per unit of surface area (cm²) and per unit of time (minute).

According to a preferred embodiment, the “volatile linear alkanes” suitable for the invention have a nonzero vapour pressure (also known as saturated vapour pressure) at ambient temperature, in particular a vapour pressure ranging from 0.3 Pa to 6000 Pa.

Preferably, the “volatile linear alkanes” suitable for the invention have a vapour pressure ranging from 0.3 to 2000 Pa, at ambient temperature (25° C.)

Preferably, the “volatile linear alkanes” suitable for the invention have a vapour pressure ranging from 0.3 to 1000 Pa at ambient temperature (25° C.)

More preferably, the “volatile linear alkanes” suitable for the invention have a vapour pressure ranging from 0.4 to 600 Pa at ambient temperature (25° C.)

Preferably, the “volatile linear alkanes” suitable for the invention have a vapour pressure ranging from 1 to 200 Pa at ambient temperature (25° C.)

More preferably, the “volatile linear alkanes” suitable for the invention have a vapour pressure ranging from 3 to 60 Pa at ambient temperature (25° C.)

According to an embodiment, a volatile linear alkane suitable for the invention can exhibit a flash point within the range varying from 30 to 120° C. and more particularly from 40 to 100° C. The flash point is in particular measured according to standard ISO 3679.

According to one embodiment, an alkane suitable for the invention can be a volatile linear alkane comprising from 7 to 14 carbon atoms.

Preferably, the “volatile linear alkanes” suitable for the invention comprise from 8 to 14 carbon atoms.

Preferably, the “volatile linear alkanes” suitable for the invention comprise from 9 to 14 carbon atoms.

Preferably, the “volatile linear alkanes” suitable for the invention comprise from 10 to 14 carbon atoms.

Preferably, the “volatile linear alkanes” suitable for the invention comprise from 11 to 14 carbon atoms.

According to an advantageous embodiment, the “volatile linear alkanes” suitable for the invention exhibit a rate of evaporation as defined above ranging from 0.01 to 3.5 mg/cm²/min at ambient temperature (25° C.) and atmospheric pressure (760 mmHg) and comprise from 8 to 14 carbon atoms.

A volatile linear alkane suitable for the invention can advantageously be of vegetable origin.

Preferably, the volatile linear alkane or the mixture of volatile linear alkanes present in the composition according to the invention comprises at least a ¹⁴C isotope of carbon (carbon-14); in particular, the ¹⁴C isotope can be present in a ¹C/¹²C ratio of greater than or equal to 1×10⁻¹⁶, preferably of greater than or equal to 1×10⁻¹⁵, more preferably of greater than or equal to 7.5×10⁻¹⁴ and better still of greater than or equal to 1.5×10⁻¹³. Preferably, the ¹⁴C/¹²C ratio ranges from 6×10⁻¹³ to 1.2×10⁻¹².

The amount of ¹⁴C isotopes in the volatile linear alkane or the mixture of volatile linear alkanes can be determined by methods known to a person skilled in the art, such as the Libby counting method, liquid scintillation spectrometry or accelerator mass spectrometry.

Such an alkane can be obtained, directly or in several stages, from a vegetable starting material, such as an oil, a butter, a wax, and the like.

Mention may be made, as example of alkanes suitable for the invention, of the alkanes described in the patent applications from Cognis, WO 2007/068371 or WO2008/155059 (mixtures of distinct alkanes differing by at least one carbon). These alkanes are obtained from fatty alcohols, themselves obtained from coconut or palm oil.

Mention may be made, as example of linear alkanes suitable for the invention, of n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄), and their mixtures. According to a specific embodiment, the volatile linear alkane is chosen from n-nonane, n-undecane, n-dodecane, n-tridecane, n-tetradecane and their mixtures.

According to a preferred form, mention may be made of the mixtures of n-undecane (C₁₁) and of n-tridecane (C₁₃) obtained in Examples 1 and 2 of Application WO2008/155059 of Cognis.

Mention may also be made of the n-dodecane (C₁₂) and the n-tetradecane (C₁₄) sold by Sasol under the references Parafol 12-97 and Parafol 14-97 respectively, and their mixtures.

The volatile linear alkane can be used alone.

Alternatively or preferentially, use may be made of a mixture of at least two distinct volatile linear alkanes differing from one another by a carbon number n of at least 1, in particular differing from one another by a carbon number of 1 or 2.

According to a first embodiment, use is made of a mixture of at least two distinct volatile linear alkanes comprising from 10 to 14 carbon atoms and differing from one another by a carbon number of at least 1. Mention may be made, as examples, in particular of the C₁₀/C₁₁, C₁₁/C₁₂ or C₁₂/C₁₃ mixtures of volatile linear alkanes.

According to another embodiment, use is made of a mixture of at least two distinct volatile linear alkanes comprising from 10 to 14 carbon atoms and differing from one another by a carbon number of at least 2. Mention may be made, as examples, in particular of the C₁₀/C₁₂ or C₁₂/C₁₄ mixtures of volatile linear alkanes, for an even carbon number n, and the C₁₁/C₁₃ mixture, for an uneven carbon number n.

According to a preferred form, use is made of a mixture of at least two distinct volatile linear alkanes comprising from 10 to 14 carbon atoms which differ from one another by a carbon number of at least and in particular of a C₁₁/C₁₃ mixture of volatile linear alkanes or a C₁₂/C₁₄ mixture of volatile linear alkanes.

Other mixtures combining more than two volatile linear alkanes according to the invention, such as, for example, a mixture of at least three distinct volatile linear alkanes comprising from 7 to 14 carbon atoms which differ from one another by a carbon number of at least 1, also come within the invention but the mixtures of two volatile linear alkanes according to the invention are preferred (binary mixtures), the said two volatile linear alkanes preferably representing more than 95% by weight and better still more than 99% by weight of the total content of volatile linear alkanes in their mixture.

According to a specific form of the invention, in a mixture of volatile linear alkanes, the volatile linear alkane having the smallest carbon number is predominant in the mixture.

According to another form of the invention, use is made of a mixture of volatile linear alkanes in which the volatile linear alkane having the greatest carbon number is predominant in the mixture.

Mention may in particular be made, as examples of mixtures suitable for the invention, of the following mixtures:
from 50 to 90% by weight, preferably from 55 to 80% by weight, more preferably from 60 to 75% by weight, of a volatile linear Cₙ alkane with n ranging from 7 to 14,
from 10 to 50% by weight, preferably from 20 to 45% by weight, preferably from 24 to 40% by weight, of volatile linear Cₙ₊ₓ alkane with x greater than or equal to 1, preferably x=1 or x=2, with n+x between 8 and 14,

with respect to the total weight of the alkanes in the said mixture.

In particular, the said mixture of alkanes according to the invention comprises:
less than 2% by weight, preferably less than 1% by weight, of branched hydrocarbons,
and/or less than 2% by weight, preferably less than 1% by weight, of aromatic hydrocarbons,
and/or less than 2% by weight, preferably less than 1% by weight and preferentially less than 0.1% by weight of unsaturated hydrocarbons in the mixture.

More particularly, a volatile linear alkane suitable for the invention can be employed in the form of an n-undecane/n-tridecane mixture.

In particular, use will be made of a mixture of volatile linear alkanes comprising:
from 55 to 80% by weight, preferably from 60 to 75% by weight, of volatile linear C₁₁ alkane (n-undecane),
from 20 to 45% by weight, preferably from 24 to 40% by weight, of volatile linear C₁₃ alkane (n-tridecane),

with respect to the total weight of the alkanes in the said mixture.

According to a specific embodiment, the mixture of alkanes is an n-undecane/n-tridecane mixture. In particular, such a mixture can be obtained according to Example 1 or Example 2 of WO 2008/155059.

According to another specific embodiment, use is made of the n-dodecane sold under the reference Parafol 12-97 by SASOL.

According to another specific embodiment, use is made of the n-tetradecane sold under the reference Parafol 14-97 by SASOL.

According to yet another embodiment, use is made of a mixture of n-dodecane and n-tetradecane.

According to a specific form of the invention, the composition can comprise less than 10% by weight, indeed even less than 5% by weight, indeed even less than 2% by weight, indeed even be devoid of cyclic silicone oil.

Formulation

The composition according to the invention, in the form of a water-in-oil emulsion, is characterized in particular embodiments by an aqueous phase dispersed in a liquid fatty phase, generally a surfactant and the presence optionally of at least one thickening agent intended to increase the viscosity of the said composition.

Aqueous Phase

A composition according to the invention comprises at least one aqueous phase, preferably in a content ranging from 10 to 80% by weight and more particularly from 25 to 70% by weight, with respect to the total weight of the composition.

The aqueous phase comprises water and/or at least one water-soluble solvent.

The water can be a floral water, such as cornflower water, and/or a mineral water, such as water from Vittel, water from Lucas or water from La Roche-Posay, and/or a thermal water.

The term “water-soluble solvent” denotes, in the present invention, a compound which is liquid at ambient temperature and which is miscible with water (miscibility in water of greater than 50% by weight at 25° C. and atmospheric pressure).

The water-soluble solvent(s) suitable for the invention can be chosen from C₁₋₈ monoalcohols, in particular C₁₋₅ monoalcohols, in particular ethanol, isopropanol, tert-butanol or n-butanol, polyols, such as described above, and their mixtures. Also very particularly suitable for the invention are ethanol and isopropanol and preferably ethanol.

A composition of the invention can additionally comprise at least one salt, for example sodium chloride, magnesium chloride and magnesium sulphate.

A composition of the invention can comprise from 0.05 to 1.5% by weight, in particular from 0.1 to 1.0% by weight and more particularly from 0.15 to 0.8% by weight of salts, with respect to the total weight of the composition.

The aqueous phase can also comprise any water-soluble or water-dispersible compound compatible with an aqueous phase, such as gelling agents, film-forming polymers, thickeners, surfactants and their mixtures.

Liquid Fatty Phase

A cosmetic composition in accordance with the present invention comprises at least one liquid fatty phase comprising at least one volatile linear alkane, in particular C₇-C₁₄ alkane, as defined above and optionally at least one additional oil and/or additional fatty substances.

A composition of the invention can preferably comprise a liquid fatty phase in a content varying from 10 to 90% by weight, in particular from 15 to 60% by weight and more particularly from 20 to 50% by weight, with respect to the total weight of the composition.

Additional Oils

A composition according to the invention can comprise at least one additional oil chosen from volatile and nonvolatile oils of hydrocarbon, silicone or fluorinated type. The oils can be of animal, vegetable, mineral or synthetic origin.

The term “oil” is understood to mean any fatty substance in the liquid form at ambient temperature (20-25° C.) and at atmospheric pressure.

The term “volatile oil” is understood to mean an oil (or nonaqueous medium) capable of evaporating on contact with the skin in less than one hour at ambient temperature and atmospheric pressure. The volatile oil is a volatile cosmetic oil which is liquid at ambient temperature and which has in particular a nonzero vapour pressure at ambient temperature and atmospheric pressure, in particular which has a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

In addition, the volatile oil generally has a boiling point, measured at atmospheric pressure, ranging from 150° C. to 260° C. and preferably ranging from 170° C. to 250° C.

Advantageously, the fatty phase comprises from 5 to 40% by weight, preferably from 10 to 35% by weight and preferentially from 15 to 30% by weight of volatile oil(s), with respect to the total weight of the composition.

The term “hydrocarbon oil” is understood to mean an oil essentially formed, indeed even composed, of carbon and hydrogen atoms and optionally of oxygen and nitrogen atoms which does not comprise a silicon or fluorine atom; it can comprise ester, ether, amine and amide groups.

The term “silicone oil” is understood to mean an oil comprising at least one silicon atom and in particular comprising Si—O groups.

The term “fluorinated oil” is understood to mean an oil comprising at least one fluorine atom.

Additional Volatile Oils

The volatile hydrocarbon oil which can be used in the invention can be chosen from hydrocarbon oils having a flash point ranging from 40° C. to 102° C., preferably ranging from 40° C. to 55° C. and preferably ranging from 40° C. to 50° C.

Mention may be made, as volatile hydrocarbon oils having from 7 to 16 carbon atoms, of in particular volatile linear C₇-C₁₄ alkanes, branched C₈-C₁₄ alkanes, such as C₈-C₁₄ isoalkanes (also known as isoparaffins), isododecane, isodecane, isohexadecane and, for example, the oils sold under the Isopar or Permethyl commercial names, branched C₈-C₁₄ esters, such as isohexyl neopentanoate, and their mixtures. Preferably, the volatile hydrocarbon oil is chosen from volatile hydrocarbon oils having from 8 to 16 carbon atoms and their mixtures, in particular from isododecane, isodecane or isohexadecane, and is in particular isododecane.

The volatile silicone oil which can be used in the invention can be chosen from silicone oils having a flash point ranging from 40° C. to 102° C., preferably having a flash point of greater than 55° C. and less than or equal to 95° C., and preferentially ranging from 65° C. to 95° C.

Mention may be made, as volatile silicone oil, of linear or cyclic silicone oils having from 2 to 7 silicon atoms, these silicones optionally comprising alkyl or alkoxy groups having from 1 to 10 carbon atoms. Mention may in particular be made, as examples of volatile silicone oil, of octamethylcyclotetra-siloxane, decamethylcyclopentasiloxane, dodecamethyl-cyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may be made, as volatile fluorinated oil, of nonafluoroethoxybutane, nonafluoromethoxybutane, decafluoropentane, tetradecafluorohexane, dodecafluoro-pentane and their mixtures.

The additional volatile oil chosen from additional volatile hydrocarbon oils, volatile silicone oils, volatile fluorinated oils and their mixtures can be present in a content ranging from 0 to 25% by weight, with respect to the total weight of the oils, in particular from 0 to 15% by weight and preferentially ranging from 0 to 10% by weight, with respect to the total weight of the composition. According to a specific form, the composition does not comprise additional volatile oil.

The fatty phase of the emulsion according to the invention can additionally comprise at least one nonvolatile oil.

Nonvolatile Oils

This nonvolatile oil or one of its mixtures can be present in a content ranging from 0 to 25% by weight, with respect to the total weight of the emulsion, and preferably ranging from 0 to 10% by weight.

The nonvolatile oil can be chosen from carbon-comprising, hydrocarbon-comprising and/or silicone oils of mineral, animal, vegetable or synthetic origin, and their mixtures, in so far as they are compatible with the use envisaged.

Mention may be made of nonvolatile hydrocarbon oils, such as liquid paraffin or liquid petrolatum, isoeicosane, soybean oil, perhydrosqualene, sweet almond, calophyllum, palm, grape seed, sesame, maize, arara, rapeseed, sunflower, cottonseed, apricot, castor, avocado, jojoba, olive or cereal germ oil; esters of lanolic acid, oleic acid, lauric acid or stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, di(2-ethylhexyl) succinate, diisostearyl malate, glyceryl triisostearate or diglyceryl triisostearate; higher fatty acids, such as myristic acid, palmitic acid, stearic acid, behenic acid, oleic acid, linoleic acid, linolenic acid or isostearic acid; or higher fatty alcohols, such as cetanol, stearyl alcohol or oleyl alcohol, linoleyl or linolenyl alcohol, isostearyl alcohol or octyldodecanol.

Mention may be made, as nonvolatile silicone oil, of polydimethylsiloxanes (PDMSs), which are optionally phenylated, such as phenyl trimethicones, or optionally substituted by aliphatic and/or aromatic groups or by functional groups, such as hydroxyl, thiol and/or amine groups; polysiloxanes modified with fatty acids, fatty alcohols or polyoxyalkylenes, and their mixtures.

According to a specific form, the composition can comprise a polydimethylsiloxane (PDMS) oil, it being understood that the volatile linear alkanes/PDMS ratio by weight will be greater than or equal to 1.

Preferably, these additional volatile or nonvolatile oils represent less than 50% by weight, in particular less than 40% by weight, indeed even less than 30% by weight, less than 20% by weight, or even less than 10% by weight, or even less than 1% by weight, with respect to the total weight of the oils in the composition.

Thus, the ratio by weight of the volatile linear C₇-C₁₆ alkanes to the additional oils will preferably be greater than or equal to 1.

A composition according to the invention will generally comprise at least one surface-active agent.

Surface-Active Agents

The surface-active agents which can be used according to the invention, suitable for W/O emulsions, are chosen in particular from amphoteric, anionic, cationic or nonionic surface-active agents, used alone or as mixtures. According to a preferred form, use will be made of nonionic surfactants.

The surface-active agents are generally present in the composition in a content which can range from 0.5 to 15% by weight, in particular from 1.5 to 5% by weight, with respect to the total weight of the composition.

By way of example, the silicone surfactant(s) can be present in a content ranging from 0.5 to 10% by weight, in particular from 1 to 5% by weight, with respect to the total weight of the composition.

According to another specific embodiment, the nonsilicone surfactant(s) can be present in a content ranging from 1 to 10% by weight, in particular from 2 to 8% by weight, with respect to the total weight of the composition.

For the W/O emulsions, use may in particular be made of hydrocarbon-comprising or silicone surface-active agents.

Mention may be made, for example, as hydrocarbon-comprising surface-active agents, of polyesters of polyols, such as PEG-30 dipolyhydroxystearate, sold under the reference Arlacel P 135 by Uniqema, or polyglyceryl-2 dipolyhydroxystearate, sold under the reference Dehymuls PGPH by Cognis.

Mention may be made, for example, as silicone surface-active agents, of alkyl dimethicone copolyols, such as lauryl dimethicone copolyol, sold under the name “Dow Corning 5200 Formulation Aid” by Dow Corning, and cetyl dimethicone copolyol, sold under the name Abil EM 90 by Goldschmidt, or the polyglyceryl-4 isostearate/cetyl dimethicone copolyol/hexyl laurate mixture sold under the name Abil WE 09 by Goldschmidt.

One or more coemulsifiers can also be added thereto. Advantageously, the coemulsifier can be chosen from the group consisting of polyol alkyl esters. Mention may in particular be made, as polyol alkyl esters, of esters of glycerol and/or of sorbitan, for example polyglyceryl-3 diisostearate, sold under the name of Lameform TGI by Cognis, polyglycerol-4 isostearate, such as the product sold under the name Isolan GI 34 by Goldschmidt, sorbitan isostearate, such as the product sold under the name Arlacel 987 by ICI, glycerol sorbitan isostearate, such as the product sold under the name Arlacel 986 by ICI, and their mixtures.

According to a specific form, the composition of the invention comprises at least one silicone surfactant chosen from dimethicone copolyols, in particular the polyglyceryl-4 isostearate/cetyl dimethicone copolyol/hexyl laurate mixture sold under the name Abil WE 09 by Goldschmidt.

The composition according to the invention can also comprise at least one gelling or structuring agent for the fatty phase intended to increase the viscosity of the said composition.

According to a specific form of the invention, the composition comprises at least one lipophilic gelling agent.

Lipophilic Gelling Agents

A gelling agent suitable for the invention is advantageously lipophilic. A lipophilic gelling agent can be inorganic or organic (in particular polymeric).

The lipophilic gelling agent or agents can be present in the composition in a content ranging from 0.1 to 20% by weight, in particular from 0.2 to 15% by weight and better still from 0.25 to 10% by weight, with respect to the total weight of the said composition.

Inorganic Gelling Agents

Mention may be made, as lipophilic gelling agents, for example, of hydrophobic modified clays, such as modified magnesium silicate (Bentone Gel VS38 from Rheox) or hectorite modified by distearyldimethyl-ammonium chloride (CTFA name: Disteardimonium hectorite), sold under the name “Bentone 38 CE” by Rheox.

Mention may be made, as inorganic lipophilic gelling agent, of optionally modified clays, such as hectorites modified by a C₁₀ to C₂₂ fatty acid ammonium chloride, such as hectorite modified by distearyl-dimethylammonium chloride, such as, for example, that sold under the name of Bentone 38V by Elementis.

Polymeric Organic Gelling Agents

The polymeric organic lipophilic gelling agents are, for example, partially or completely crosslinked organopolysiloxane elastomers, of three-dimensional structure, such as those sold under the names of KSG6®, KSG16® and KSG18® by Shin Etsu, of Trefil E-505C® and Trefil E-506C® by Dow Corning, of Gransil SR-CYC®, SR DMF10®, SR-DC556®, SR 5CYC Gel®, SR DMF 10 Gel® and SR DC 556 Gel® by Grant Industries, or of SF 1204® and JK 113® by General Electric; ethylcellulose, such as that sold under the name Ethocel® by Dow Chemical; polycondensates of polyamide type resulting from the condensation between a dicarboxylic acid comprising at least 32 carbon atoms and an alkylenediamine, in particular ethylenediamine, in which the polymer comprises at least one end carboxylic acid group esterified or amidated with at least one monoalcohol or one monoamine comprising from 12 to 30 carbon atoms which are linear and saturated, in particular ethylenediamine/stearyl dilinoleate copolymers, such as that sold under the name Uniclear 100 VG® by Arizona Chemical; galactomannans comprising from 1 to 6 and in particular from 2 to 4 hydroxyl groups per monosaccharide which are substituted by a saturated or unsaturated alkyl chain, such as guar gum alkylated by C₁ to C₆ and in particular C₁ to C₃ alkyl chains, and their mixtures.

Mention may also be made, as lipophilic gelling agents suitable for the invention, of copolymers of the polystyrene/polyalkylene type and more particularly block copolymers of “diblock”, “triblock” or “radial” type of the polystyrene/polyisoprene or polystyrene/polybutadiene type, such as those sold under the name Luvitol HSB® by BASF, of the polystyrene/copoly(ethylene-propylene) type, such as those sold under the name of Kraton® by Kraton Polymers, or of the polystyrene/copoly(ethylene-butylene) type, or blends of triblock and radial (star) copolymers in isododecane, such as those sold by Penreco under the name Versagel®, such as, for example, the blend of butylene/ethylene/styrene triblock copolymer and of ethylene/propylene/styrene star copolymer in isododecane (Versagel M 5960).

Mention may also be made, among the lipophilic gelling agents which can be used in a cosmetic composition of the invention, of esters of dextrin and of fatty acid, such as dextrin palmitates, in particular such as those sold under the names Rheopearl TL® or Rheopearl KL® by Chiba Flour, hydrogenated vegetable oils, such as hydrogenated castor oil, or fatty alcohols, in particular C₈ to C₂₆ and more particularly C₁₂ to C₂₂ fatty alcohols, such as, for example, myristyl alcohol, cetyl alcohol, stearyl alcohol or behenyl alcohol.

According to a particularly preferred embodiment, a composition according to the invention can comprise at least one inorganic lipophilic gelling agent chosen from hydrophobic modified clays.

Lipophilic Structuring Agents

The fatty phase can also comprise at least one lipophilic structuring agent. The lipophilic structuring agent or agents can be present in the composition in a content ranging from 0.05 to 10% by weight, in particular from 0.1 to 8% by weight and better still from 0.2 to 5% by weight, with respect to the total weight of the said composition.

In particular, the said lipophilic structuring agent can be chosen from at least one wax, at least one gum and/or at least one pasty fatty substance of vegetable, animal, mineral or synthetic origin, indeed even silicone origin, and their mixtures.

Waxes

Mention may be made, among waxes which are solid at ambient temperature and which are capable of being present in the composition according to the invention, of hydrocarbon waxes, such as beeswax, carnauba, candelilla, ouricury or Japan wax, cork fibre or sugarcane waxes, paraffin or lignite waxes, micro-crystalline waxes, lanolin wax, montan wax, ozokerites, polyethylene waxes, the waxes obtained by the Fischer-Tropsch synthesis, hydrogenated oils, or fatty esters and glycerides which are solid at 25° C. Use may also be made of silicone waxes, among which may be mentioned alkylpolymethylsiloxanes, alkoxypolymethylsiloxanes and/or polymethylsiloxane esters. The waxes can be provided in the form of stable dispersions of colloidal wax particles, such as can be prepared according to known methods, such as those of “Microemulsions Theory and Practice”, edited by L. M. Prince, Academic Press (1977), pages 21-32. Mention may be made, as wax which is liquid at ambient temperature, of jojoba oil.

Mention may also be made of hydrogenated vegetable oils, such as hydrogenated castor oil.

Mention may be made, as lipophilic structuring agent also suitable for the invention, of fatty alcohols, in particular C₈ to C₂₆ and more particularly C₁₂ to C₂₂ fatty alcohols.

According to one embodiment, a fatty alcohol suitable for the invention can be chosen from myristyl alcohol, cetyl alcohol, stearyl alcohol or behenyl alcohol.

Mention may be made, as lipophilic structuring agent also suitable for the invention, of esters of fatty acids and glycerols, such as glyceryl tristearate.

The waxes can be present in a proportion of 0.1 to 10% by weight, with respect to the total weight of the emulsion, preferably of 0.1 to 5% by weight.

Pasty Fatty Compounds

Pasty fatty compounds can be defined using at least one of the following physicochemical properties:
a viscosity of 0.1 to 40 Pa·s (1 to 400 poises), preferably 0.5 to 25 Pa·s, measured at 40° C. with a Contraves TV rotary viscometer equipped with an MS-r3 or MS-r4 spindle at a frequency of 60 Hz,
a melting point of 25-70° C., preferably 25-55° C.

The compositions of the invention can also comprise at least one alkyl, alkoxy or phenyl dimethicone, such as, for example, the product sold under the name of “Abil Wax 2440” by Goldschmidt.

The composition of the invention can also comprise at least one filler of organic or inorganic nature.

Fillers

The term “filler” should be understood as meaning colourless or white solid particles of any shape which are provided in a form insoluble and dispersed in the medium of the composition. Of inorganic or organic nature, they make it possible to confer, on the composition, softness, mattness and uniformity on making-up.

The fillers can be present in the emulsion in a content ranging from 0.1 to 10% by weight, with respect to the total weight of the emulsion, preferably from 0.5 to 7% by weight.

The fillers used in the compositions according to the present invention can be lamellar, globular or spherical in form, in the form of fibres or in any other form intermediary between these defined forms.

The fillers according to the invention may or may not be surface-coated and, in particular, they may be treated at the surface with silicones, amino acids, fluorinated derivatives or any other substance promoting the dispersion and the compatibility of the filler in the composition.

Mention may be made, among inorganic fillers which can be used in the compositions according to the invention, of talc, mica, silica, trimethylsiloxy-silicate, kaolin, bentone, calcium carbonate and magnesium hydrogencarbonate, hydroxyapatite, boron nitride, hollow silica microspheres (Silica Beads from Maprecos), glass or ceramic microcapsules, silica-based fillers, such as Aerosil 200 or Aerosil 300; Sunsphere H-33 or Sunsphere H-51, sold by Asahi Glass; Chemicelen, sold by Asahi Chemical; composites of silica and of titanium dioxide, such as the TSG series sold by Nippon Sheet Glass, and their mixtures.

Mention may be made, among the organic fillers which can be used in the compositions according to the invention, of powders formed of polyamide (Nylon® Orgasol from Atochem), of poly-β-alanine and of poly-ethylene, powders formed of polytetrafluoroethylene (Teflon®), lauroyllysine, starch, powders formed of tetrafluoroethylene polymers, hollow microspheres of polymers, such as Expancel (Nobel Industrie), metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, Polypore® L 200 (Chemdal Corporation), silicone resin microbeads (Tospearl® from Toshiba, for example), polyurethane powders, in particular powders formed of crosslinked polyurethane comprising a copolymer, the said copolymer comprising trimethylol hexyllactone, such as the hexamethylene diisocyanate/trimethylol hexyllactone polymer sold under the name of Plastic Powder D-400® or Plastic Powder D-800® by Toshiki, carnauba microwaxes, such as that sold under the name of MicroCare 350® by Micro Powders, synthetic wax microwaxes, such as that sold under the name of MicroEase 114S® by Micro Powders, the microwaxes composed of a mixture of carnauba wax and of polyethylene wax, such as those sold under the names of MicroCare 300® and 310® by Micro Powders, the microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, or fibres of synthetic or natural and inorganic or organic origin. They can be short or long, individual or organized, for example braided, or hollow or solid. They can have any shape and can in particular be circular or polygonal (square, hexagonal or octagonal) in cross section, according to the specific application envisaged. In particular, their ends are blunted and/or polished in order to avoid inflicting injury. The fibres have a length ranging from 1 μm to 10 mm, preferably from 0.1 mm to 5 mm and better still from 0.3 mm to 3 mm. Their cross section can be included within a circle with a diameter ranging from 2 nm to 500 μm, preferably ranging from 100 nm to 100 μm and better still from 1 μm to 50 μm. Mention may be made, as fibres which can be used in the compositions according to the invention, of nonrigid fibres, such as polyamide (Nylon®) fibres, or rigid fibres, such as poly(imide-amide) fibres, for example those sold under the names Kermel® or Kermel Tech® by Rhodia, or poly(p-phenylene terephthalamide) (or aramid) fibres, in particular sold under the name Kevlar® by DuPont de Nemours,

and their mixtures.

Cosmetic Adjuvants

The compositions of the invention can additionally comprise one or more of the adjuvants known in the cosmetic and dermatological fields, such as moisturizing agents; emollients; hydrophilic or lipophilic active principles; agents for combating free radicals; sequestering agents; antioxidants; preservatives; basifying or acidifying agents; fragrances; hydrophilic gelling agents; film-forming agents, in particular film-forming polymers (for compositions in a sustained direction); organic or physical sunscreens; water-soluble or fat-soluble dyes; and their mixtures. The amounts of these various adjuvants are those conventionally used in foundations.

Of course, a person skilled in the art will take care to choose the optional adjuvant or adjuvants added to the composition according to the invention so that the advantageous properties intrinsically attached to the composition in accordance with the invention are not, or not substantially, detrimentally affected by the envisaged addition.

These adjuvants are generally present in the composition in a content ranging from 0.01 to 20% by weight, preferably from 0.1 to 10% by weight, with respect to the total weight of the said composition.

The invention is illustrated in the examples presented below by way of illustration and without implied limitation of the scope of the invention:

Unless otherwise indicated, the values in the examples below are expressed in % by weight, with respect to the total weight of the composition.

### EXAMPLES

### Example 1

### Cosmetic Properties of the Compositions According to the Invention

a) Composition and Preparation of the Test Formulae

| | Formula A | Formula B | Formula C |
|---|---|---|---|
| Ingredients | (comparative) | (invention) | (invention) |
| Magnesium sulphate | 1.50 | 1.50 | 1.50 |
| Hectorite modified by | 1 | 1 | 1 |
| distearyldimethyl- | | | |
| ammonium | | | |
| (Bentone 38 VCG from | | | |
| Elementis) | | | |
| Microspheres (vinylidene | 0.60 | 0.60 | 0.60 |
| chloride/acrylonitrile/ | | | |
| methyl | | | |
| methacrylate copolymer) | | | |
| expanded with isobutane | | | |
| (Expancel 551 DE 40 D42 | | | |
| from Expancel) | | | |
| Titanium dioxide | 21.20 | 21.20 | 21.20 |
| Red iron oxide | 0.89 | 0.89 | 0.89 |
| Black iron oxide | 0.33 | 0.33 | 0.33 |
| Yellow iron oxide | 2.58 | 2.58 | 2.58 |
| Tristearin acetylated glycol | 0.30 | 0.30 | 0.30 |
| stearate (Unitwix from | | | |
| United Guardian) | | | |
| Sodium carboxymethyl- | 0.50 | 0.50 | 0.50 |
| cellulose | | | |
| Cyclopentasiloxane | 15.30 | | |
| Polyglyceryl-4 | 9 | 9 | 9 |
| isostearate/cetyl PEG/PPG- | | | |
| 10/1/hexyl laurate (Abil | | | |
| WE 09 from Evonik | | | |
| Goldschmidt) | | | |
| Polydimethylsiloxane | 6.60 | 6.60 | |
| (viscosity: 5 cSt) (Xiameter | | | |
| PMX-200 Silicone Fluid | | | |
| 5CS from Dow Corning) | | | |
| Glycerol | 5 | 5 | 5 |
| Water from La Roche- | 31.70 | 31.70 | 31.70 |
| Posay | | | |
| Ethylhexylglycerin | 0.50 | 0.50 | 0.50 |
| Pentylene glycol | 3 | 3 | 3 |
| Predominant mixture of | | 15.30 | 21.90 |
| n-undecane:n-tridecane in | | | |
| which the n-undecane is | | | |
| predominant in the | | | |
| mixture* | | | |

*as prepared according to Application WO2008/155059

The compositions are prepared as follows:

The mixture of surfactants is heated on a water bath in a beaker at 75° C. Once the mixture has melted (approximately 65° C.), it is placed on a heating plate (set value 100° C.) and stirred with a turbine.

The pigments are formed into a paste with a portion of the fatty phase and passed three times through a triple roll mill. The remainder of the fatty phase is used to form the fillers into a paste.

At 65° C., the bentone is added to the mixture of surfactants heated and subjected to turbine mixing, the pigment mixture and the filler in paste form are added at 40° C. and the mixture is left to cool under a Moritz mixer.

The mixture is allowed to cool at 25° C.

At the same time, all the constituents of the aqueous phase are mixed, the combined product is weighed and the water is added, and then the mixture is placed on a heating plate under a Rayneri mixer to reach a temperature of 70° C. The cellulose is sprinkled over it and stirring is carried until complete dissolution is achieved. Emulsification is carried out at ambient temperature for 10 min under a turbine binding the aqueous phase to the preprepared fatty phase.

The same emulsion was prepared with 30% of pigments (iron oxides+titanium dioxide) instead of 25%, respectively with a volatile silicon oil (formula D) and with at least one volatile linear alkane according to the invention (formula E).

| | Formula D | Formula E |
|---|---|---|
| Ingredients | (comparative) | (invention) |
| Magnesium sulphate | 1.50 | 1.50 |
| Hectorite modified by | 1 | 1 |
| distearyldimethylammonium | | |
| (Bentone 38 VCG from Elementis) | | |
| Microspheres (vinylidene | 0.60 | 0.60 |
| chloride/acrylonitrile/methyl | | |
| methacrylate copolymer) expanded | | |
| with isobutane (Expancel 551 DE | | |
| 40 D42 from Expancel) | | |
| Titanium dioxide | 25.06 | 25.06 |
| Red iron oxide | 1.16 | 1.16 |
| Black iron oxide | 0.43 | 0.43 |
| Yellow iron oxide | 3.35 | 3.35 |
| Tristearin acetylated glycol | 0.30 | 0.30 |
| stearate (Unitwix from United | | |
| Guardian) | | |
| Sodium carboxymethylcellulose | 0.50 | 0.50 |
| Cyclopentasiloxane | 16.9 | |
| Polyglyceryl-4 isostearate/cetyl | 9 | 9 |
| PEG/PPG-10/1/hexyl laurate (Abil | | |
| WE 09 from Evonik Goldschmidt) | | |
| Polydimethylsiloxane (viscosity: | 0 | 0 |
| 5 cSt) (Xiameter PMX-200 Silicone | | |
| Fluid 5CS from Dow Corning) | | |
| Glycerol | 5 | 5 |
| Water from La Roche-Posay | 30.70 | 30.70 |
| Ethylhexylglycerin | 0.50 | 0.50 |
| Pentylene glycol | 3 | 3 |
| Predominant mixture of | | 16.9 |
| n-undecane:n-tridecane in which | | |
| the n-undecane is predominant in | | |
| the mixture* | | |

*as prepared according to Application WO2008/155059

b) Stability

The stability of the compositions A to E is evaluated by macroscopic and microscopic observation after 24 hours, one month and two months at ambient temperature, 45° C. and after three temperature cycles from −20° C. to +20° C. in an oven (for example: Vötch VT4004).

Each of the cycles lasts 24 hours and comprises the following stages: 6 hours at 20° C., then 6 hours of temperature decline down to −20° C., then 6 hours at a temperature of −20° C. and, finally, 6 hours of temperature rise up to 20° C.

After each cycle, the macroscopic and microscopic appearances of the composition are evaluated.

After three cycles, the composition must not exhibit modifications in macroscopic appearance; it must remain smooth and homogeneous, without release, without phase separation and without change in colour.

The composition is observed under a microscope between microscope slide and coverglass at a magnification ×10. Its microscopic appearance must remain close to the initial aspect: in particular, decomposition of the emulsion must not be observed (emulsion base coarser, coalescence reflected by the presence of numerous large drops, modification of the preparation edges, presence of crystals).

The test is regarded as stable if the emulsion remains fine, without release over the edges and without coalescence.

The composition A (comparative) is unstable after three temperature cycles.

The compositions B and C (invention) are stable after 24 h, one month and two months.

The composition D (comparative) results in a nonhomogeneous mixture of pigments, aqueous phase and oily phase, whereas the composition E (invention) remains stable after 24 h, one month and two months.

c) Sensory Evaluation

Protocol

Furthermore, a sensory evaluation of the said compositions A to C was carried out on a panel of 15 experts who are users of foundation. After standardized application, each person evaluates the perception of the said composition at the time of the application and in terms of makeup result according to certain predefined criteria on a standardized scale.

On application, each model evaluates in particular the following parameters: fluidity, slip, powdery feel, ease of application and greasy feel.

After application, each model evaluates in particular the makeup result: powdery finish, coverage and uniformity.

Results

Compared with the formula A (comparative), the emulsions B and C obtained are creamy, easy to apply, nongreasy, make it possible to cover very marked skin imperfections and leave a light, soft and nonsticky film after application.

### Example 2

### Coverage Properties of the Compositions According to the Invention

The substrates used for the measurement of coverage of the formulae A to C described above are Bioskin sheets white and black in colour (supplier Maprecos). The compositions A to C prepared in Example 1 are applied with a fingerstall at a rate of 32 mg to a surface with a diameter of 5 cm and are then dried for 90 minutes. In particular, the thickness applied is 15 μm.

The reflectance spectra are acquired using a Minolta 3700-d spectrocolorimeter (diffuse measurement geometry and D65/10° observation, specular component mode excluded) on black and white backgrounds (three measurements on each application).

The spectra are expressed as colorimetric coordinates in the CIE Yxy 1931 space within the meaning of the International Commission on Illumination.

The contrast ratio CR, or coverage, is calculated by taking the arithmetic mean of Y on the black background, dividing by the mean of Y on the white background and multiplying by 100.

CR=[Y(black background)/Y(white background)]×100

Formula A (comparative): CR=86
Formula B (comprising 15.3% of volatile linear alkane): CR=81
Formula C (comprising 21.9% of volatile linear alkane): CR=76

The three formulae are within the range of very high coverage as they are close to 100.

Advantageously, the compositions B and C according to the invention give a more natural makeup result than the composition A (comparative): the lines are less marked and the imperfections in relief (such as scales, dilated pores or dry areas) are less visible.

These results show that the use of volatile linear C₁-C₁₄ alkanes according to the invention in a WO architecture makes it possible to incorporate a high content of pigments (25 and 30% tested) with very high coverage properties while conferring, on the product, improved cosmetic properties on application in comparison with the same architecture but with volatile silicone oils.

### Example 3

### Complexion Corrector

| | Formula F |
|---|---|
| Ingredients | (invention) |
| Magnesium sulphate | 1.50 |
| Hectorite modified by | 1 |
| distearyldimethylammonium | |
| (Bentone 38 VCG from | |
| Elementis) | |
| Microspheres (vinylidene | 0.60 |
| chloride/acrylonitrile/methyl | |
| methacrylate copolymer) | |
| expanded with isobutane | |
| (Expancel 551 DE 40 D42 from | |
| Expancel) | |
| Titanium dioxide | 21.20 |
| Red iron oxide | 0.89 |
| Black iron oxide | 0.33 |
| Yellow iron oxide | 2.58 |
| Tristearin acetylated glycol | 0.30 |
| stearate (Unitwix from United | |
| Guardian) | |
| Sodium carboxymethylcellulose | 0.50 |
| Polyglyceryl-4 | 9 |
| isostearate/cetyl PEG/PPG- | |
| 10/1/hexyl laurate (Abil WE | |
| 09 from Evonik Goldschmidt) | |
| Glycerol | 5 |
| Water from La Roche-Posay | 31.70 |
| Ethylhexylglycerin | 0.50 |
| Pentylene glycol | 3 |
| Dodecane (Parafol C12-97 from | 21.9 |
| Sasol) | |

The composition F derives from the composition C according to the invention in which the predominant mixture of undecane:tridecane has been replaced by another volatile linear alkane according to the invention, dodecane.

The compositions according to the invention are applied uniformly to the skin of the face or in small dabs in order to conceal skin imperfections, in particular colour defects of the skin.

### Example 4

### Effect of the Volatile Linear Alkanes on the Cosmetic Properties of the Compositions According to the Invention in Comparison with Other Volatile Hydrocarbon Oils

The 3 following compositions are prepared as described in Example 1. The composition C (invention) is described in Example 1 and the compositions G and H (comparative) comprise volatile hydrocarbon oils other than the volatile linear alkanes according to the invention.

| | Formula C | Formula G | Formula H |
|---|---|---|---|
| Ingredients | (invention) | (comparative) | (comparative) |
| Magnesium sulphate | 1.50 | 1.50 | 1.50 |
| Hectorite modified by | 1 | 1 | 1 |
| distearyldimethyl- | | | |
| ammonium | | | |
| (Bentone 38 VCG from | | | |
| Elementis) | | | |
| Microspheres (vinylidene | 0.60 | 0.60 | 0.60 |
| chloride/acrylonitrile/ | | | |
| methyl methacrylate | | | |
| copolymer) expanded | | | |
| with isobutane | | | |
| (Expancel 551 | | | |
| DE 40 D42 from | | | |
| Expancel) | | | |
| Titanium dioxide | 21.20 | 21.20 | 21.20 |
| Red iron oxide | 0.89 | 0.89 | 0.89 |
| Black iron oxide | 0.33 | 0.33 | 0.33 |
| Yellow iron oxide | 2.58 | 2.58 | 2.58 |
| Tristearin acetylated | 0.30 | 0.30 | 0.30 |
| glycol stearate (Unitwix | | | |
| from United Guardian) | | | |
| Sodium | 0.50 | 0.50 | 0.50 |
| carboxymethylcellulose | | | |
| Isohexadecane | | 21.90 | |
| Isododecane | | | 21.90 |
| Polyglyceryl-4 iso- | 9 | 9 | 9 |
| stearate/cetyl PEG/PPG- | | | |
| 10/1/hexyl laurate (Abil | | | |
| WE 09 from Evonik | | | |
| Goldschmidt) | | | |
| Glycerol | 5 | 5 | 5 |
| Water from La Roche- | 31.70 | 31.70 | 31.70 |
| Posay | | | |
| Ethylhexylglycerin | 0.50 | 0.50 | 0.50 |
| Pentylene glycol | 3 | 3 | 3 |
| Predominant mixture of | 21.90 | | |
| n-undecane:n-tridecane | | | |
| in which the n-undecane | | | |
| is predominant in the | | | |
| mixture* | | | |

*as prepared according to Application WO2008/155059

The viscosities of the compositions thus prepared were measured and the stability and the sensory aspect of each were evaluated according to the respective protocols described in Example 1 above.

Viscosity

The viscosities of the compositions are measured, after standing for 24 h, on a Rheomat 180 (Lamy) with an M3 spindle at 25° C. after shearing at 200 rev/min for 10 min.

The following viscosities were obtained:

Composition C (invention): 25 uD, i.e. 8.6 poises

### Composition G

(isohexadecane comparative): 51 uD, i.e. 19.5 poises

### Composition H

(isododecane comparative): 31 uD, i.e. 11.1 poises

Stability

No significant difference in terms of stability is observed between these 3 compositions.

Sensory Evaluation

On the other hand, the sensory evaluation on models of these 3 compositions is greatly in favour of the composition C according to the invention. This is because:
the composition G (comparative with isohexadecane) exhibits a viscosity twice as great in comparison with the composition C (according to the invention), which makes it more difficult to apply it as the product is more pasty and problematic to spread. The layer deposited on the skin is furthermore sticky with the composition G, in contrast to the deposited layer with the composition C according to the invention.
the composition H (comparative with isododecane) exhibits a slightly greater viscosity than that of the composition C; it is easily applied at the very start but, in view of the high volatility of isododecane, the ‘playtime’ of the product (time available to the user on application to work the composition and give uniformity to the makeup rendering) is very short, not making it possible to obtain a homogeneous deposited layer after evaporation of the isododecane: the deposited layer is as set. As for the composition G, the layer deposited on the skin is furthermore sticky, in contrast to the deposited layer with the composition C according to the invention.

These results confirm that the use of the volatile linear alkanes in a water-in-oil emulsion comprising a high level of pulverulent colouring materials makes it possible to obtain a creamy texture which is easy to apply, making it possible to homogeneously cover the skin imperfections while leaving a light and nonsticky film after application, in comparison with the same composition architectures with other volatile hydrocarbon oils (for example: isododecane or isohexadecane).

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A coloured composition in the form of a water-in-oil emulsion, comprising water and:
(i) at least 18% by weight of at least one pulverulent colouring material, with respect to the total weight of the composition, and
(ii) at least one volatile linear alkane.

2. The composition according to claim 1, comprising at least 20% by weight of pulverulent colouring material with respect to the total weight of the said composition.

3. The composition according to claim 1, comprising at least 50% by weight of volatile linear alkane(s) with respect to the total content of volatile oil(s) of the composition.

4. The composition according to claim 1, comprising at least 90% by weight of volatile linear alkane(s) with respect to the total content of volatile oil(s).

5. The composition according to claim 1, wherein the said volatile linear alkane comprises from 7 to 14 carbon atoms.

6. The composition according to claim 1, wherein the at least one volatile linear alkane is chosen from n-heptane, n-octane, n-nonane, n-undecane, n-dodecane, n-tridecane, n-tetradecane and their mixtures.

7. The composition according to claim 6, comprising n-dodecane.

8. The composition according to claim 1, comprising at least two distinct volatile linear alkanes, differing from one another by a carbon number n of at least 1.

9. The composition according to claim 1, comprising a mixture of at least two volatile linear alkanes comprising
from 50 to 90% by weight of a volatile linear Cₙ alkane with n ranging from 7 to 14,
from 10 to 50% by weight of a volatile linear Cₙ₊ₓ alkane with x greater than or equal to 1,
with respect to the total weight of the volatile linear alkanes.

10. The composition according to claim 9, comprising an n-undecane:n-tridecane (C₁₁/C₁₃) mixture comprising
from 55 to 80% by weight of volatile linear C₁₁ alkane (n-undecane),
from 20 to 45% by weight of volatile linear C₁₃ alkane (n-tridecane),
with respect to the total weight of the volatile linear alkanes.

11. The composition according to claim 1, comprising from 5 to 50% by weight of volatile linear alkane(s) with respect to the total weight of the composition.

12. The composition according to claim 1, further comprising at least one silicone surfactant.

13. The composition according to claim 1, wherein it is a foundation or a complexion corrector.

14. A method for making up a keratinous substance, comprising application to the keratinous substance of a composition as defined in claim 1.

15. The method according to claim 14, wherein said method is a method for concealing a skin imperfection, comprising applying a skin imperfection-concealing amount of the composition as defined in claim 1 to skin having a skin imperfection.
